(19) United States
(12) Patent Application Publication
(10) Pub. No.: US 2024/0226059 A1
(43) Pub. Date: Jul. 11, 2024

(54) COMPOSITION FOR INHIBITING ANGIOGENESIS AND ENHANCING THERAPEUTIC EFFECT OF ANTICANCER DRUGS IN CANCER PATIENT

(71) Applicant: KANT SCIENCE INC., Incheon (KR)

(72) Inventors: Jong Doo Lee, Incheon (KR); So Won Kim, Incheon (KR); Yongchel Ahn, Incheon (KR); Sung Hae Kim, Incheon (KR)

(21) Appl. No.: 18/267,205

(22) PCT Filed: Dec. 15, 2021
(86) PCT No.: PCT/KR2021/019030

§ 371 Date: Dec. 26, 2023

(30) Foreign Application Priority Data
Dec. 15, 2020 (KR) 10-2020-0175033

(51) Int. Cl. A61K 31/353; A61K 31/05; A61K 31/122; A61K 45/06

(52) U.S. Cl. CPC A61K 31/353; A61K 31/05; A61K 31/122; A61K 45/06

(57) ABSTRACT

The present invention relates to a composition that can suppress angiogenesis and overcome the resistance of cancer cells occurring in a low-carbohydrate state, and thus can be used for angiogenesis suppression and adjuvant anticancer treatment.

### TECHNICAL FIELD

The present invention relates to a dietary composition capable of increasing the therapeutic effect of anticancer drugs by suppressing angiogenesis and overcoming the resistance of cancer cells occurring in a low-carbohydrate state.

### BACKGROUND ART

Cancer is one of the intractable diseases that all mankind must overcome, and is one of the major causes of overall mortality in Korea too. Although progress has been continuously made by regulating the cell cycle and cell death or seeking new targets including supression of oncogenes or activating cancer suppressor genes in cancer patients, the incidence of cancer is still on the rise. Currently, although many new approaches for surgical resection, chemotherapy, or radiation therapy, have been developed for cancer treatment, surgical resection is mainly effective only when cancer is diagnosed at an early stage, and it is useless when the cancer has progressed to the terminal stage, and there is no choice but to treat it mainly by using radiotherapy or chemotherapy. Chemotherapy, which was introduced to cancer treatment in the 1940s, has the advantage of being able to be relatively easily applied regardless of the stage of cancer, and thus is currently attracting a lot of attention, and various anticancer chemotherapy drugs are being developed. However, when anticancer drugs are repeatedly administered for a long period of time or when cancer recurs, there is a disadvantage in that cancer cells acquire resistance to the anticancer drugs, resulting in a loss of therapeutic effect.

Meanwhile, it is known that the ketogenic diet, such as a low-carbohydrate diet with high-fat component, is known to be effective for suppressing the growth of cancer cells. However, since cancer cells that survived long-term energy deprivation status increase in malignant potential and acquire resistance to anticancer drugs, the long-term ketogenic diet has the risk of reducing cancer-suppressing effects or even inducing cancer proliferation compared to a normal diet. Therefore, there is a need for methods for overcoming the resistance of cancer cells to low-carbohydrate diets.

### DISCLOSURE

### Technical Problem

Under the circumstances described above, the present inventors have studied methods for suppressing the resistance of cancer cells to a low-carbohydrate diet, and as a result, confirmed that the combined intake of curcumin or other natural compounds which can inhibit sodium proton exchanger 1 (NHE1) with anticancer drugs can enhance the sensitivity of anticancer drugs. Further, the present inventors confirmed that the mechanism of action of such effects tends to be closely related to hypoxia-inducible factor-1 alpha (HIF-1α) expression suppression, which promotes changes in intracellular acidity and angiogenesis.

Therefore, an object of the present invention is to provide a composition capable of enhancing anti-cancer therapeutic effects by enhancing the effects of anti-angiogenesis through regulation of microenvironmental pH and low-carbohydrate supply.

### Technical Solution

To achieve the above object, one aspect of the present invention provides a composition for suppressing intracellular alkalinization using Na+/H+exchanger type 1 (NHE1) inhibiting bioactive natural chemicals, and angiogenesis, containing one or more materials selected from the group consisting of curcumin, quercetin, catechins, resveratrol, antocyanins, isoflavones, or derivatives that suppressing NHE1 thereof.

The present inventors confirmed that when cancer cells were treated with cariporide, which is a Na+/H+exchanger type 1 (NHE1) selective inhibitor, under low-carbohydrate conditions in combination with anticancer drugs, cancer cells maintained normal pH and curcumin showed the same effect as cariporide (FIGS. 1 and 2). Curcumin is an NHE-1 inhibitor like cariporide.

In addition, it was confirmed that the anticancer effect of anticancer drugs increased (FIG. 3) and anticancer drug resistance of cancer cells decreased (FIG. 4). These effects consequentially induced the suppression of hypoxia-inducible factor-1 alpha (HIF-1α) expression (FIG. 5), and the present invention has technical significance in finding a method capable of optimally killing cancer cells under low-carbohydrate conditions.

HIF-1α is a transcription factor induced by hypoxia and consists of HIF-1α and HIF-1β. HIF-1α protein may also be accumulated by the activation of an epidermal growth factor, heregulin, a growth factor such as insulin-like growth factor-I and insulin-like growth factor-II, or an oncogene such as ErbB2 regardless of oxygen concentration. When these growth factors bind to their receptors, the synthesis of HIF-1α protein is increased, resulting in the accumulation of HIF-1α protein. HIF-1 migrating to the nucleus binds to a hypoxia responsive element (HRE) on the promoter of a target gene to induce the expression of the gene, and examples of a gene regulated by HIF-1 include a glycolytic enzyme, a vascular endothelial growth factor (VEGF), hexokinase2, a glucose transporter, erythropoietin, and the like. Due to the overexpression of these genes, cancer cells acquire traits such as resistance to cell death (apoptosis), an increase in angiogenesis ability, an increase in cell proliferation ability, an increase in invasion ability, and anticancer drug resistance to ultimately become malignant, and acquire the ability to metastasize to other tissues. Since HIF-1 plays an important role in the growth, proliferation and malignant transformation of cancer, particularly, solid tumors, as described above, research has been actively conducted to develop anticancer drugs targeting HIF-1 (Cancer Res. 2002, Vol. 62, 4316).

As already described, when the expression/activity of HIF-1α is suppressed during the treatment of cancer cells under low-carbohydrate conditions with anticancer drugs, the effect of anticancer drugs increases (FIG. 5). Therefore, the composition for suppressing angiogenesis of the present invention may be administered in combination with an anticancer drug, and may be administered while feeding a low-carbohydrate diet to a cancer patient.

These anticancer drugs may be selected from the group consisting of, but are not limited thereto, irinotecan, paclitaxel, doxorubicin, cisplatin, docetaxel, cabazitaxel, carboplatin, etoposide, gemcitabine, 5-fluorouracil, capecitabine, gefitinib, sorafenib, and imatinib, and may be preferably irinotecan or docetaxel or immune check point inhibitors.

Another aspect of the present invention provides a pharmaceutical composition for adjuvant anticancer treatment, containing one or more materials selected from the group consisting of curcumin, quercetin, catechins, resveratrol, antocyanins, isoflavones, or derivatives thereof.

As described above, when cancer cells are cultured under low-carbohydrate conditions for a long period of time, cancer cells acquire resistance, but in the present invention, when cancer cells are treated with an anticancer drug without acquiring resistance, the expression/activity of HIF-1α is further suppressed, and the effect of the anticancer drug also increases (FIG. 5). Therefore, the pharmaceutical composition for adjuvant anticancer treatment may suppress the anticancer drug resistance of cancer cells which develop in a low-carbohydrate state.

In the present invention, the composition may be used for the treatment of cancer selected from the group consisting of, but not limited thereto, colorectal cancer, breast cancer, lung cancer, bone cancer, pancreatic cancer, skin cancer, oral cancer, oropharyngeal cancer, uterine cancer, ovarian cancer, rectal cancer, gastric cancer, endometrial carcinoma, cervical carcinoma, vaginal carcinoma, small bowel cancer, thyroid cancer, parathyroid cancer, prostate cancer, chronic or acute leukemia, lymphocytic lymphoma, bladder cancer, renal cancer, liver cancer and brain tumors.

Since the physiologically active material having HIF-1α inhibitory activity as described in the composition for suppressing angiogenesis described above, and the like are used in the pharmaceutical composition for adjuvant anticancer treatment of the present invention, the description of the contents commonly shared between the two is omitted in order to avoid excess complexity of the present specification.

The pharmaceutical composition of the present invention may include a pharmaceutically acceptable carrier in addition to the active ingredient. In this case, the pharmaceutically acceptable carrier is one typically used during formulation, and examples thereof include lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, mineral oil, and the like, but are not limited thereto. Furthermore, the pharmaceutically acceptable carrier may further include a lubricant, a wetting agent, a sweetening agent, a flavoring agent, an emulsifier, a suspending agent, a preservative, and the like in addition to the above components.

The pharmaceutical composition of the present invention may be administered orally or parenterally (for example, skin application, intravenous, subcutaneous, intraperitoneal injection or applied topically) according to the target method, but is preferably orally administered.

When the active ingredient of the present invention is formulated into a preparation such as tablets, capsules, chewable tablets, a powder, a liquid, and a suspending agent for the purpose of oral administration, it is possible to include a binder such as arabic rubber, corn starch, microcrystalline cellulose or gelatin, an excipient such as calcium diphosphate or lactose, a disintegrant such as alginic acid, corn starch, or potato starch, a lubricant such as magnesium stearate, a sweetening agent such as sucrose or saccharin, and a flavoring agent such as peppermint, methyl salicylate, or a fruit flavor.

The pharmaceutical composition of the present invention is administered in a pharmaceutically effective amount. In the present invention, the ‘pharmaceutically effective amount’ refers to an amount sufficient to treat diseases at a reasonable benefit/risk ratio applicable to medical treatment, and an effective dosage level may be determined according to factors including types of diseases of patients, the severity of a disease, the activity of drugs, sensitivity to drugs, administration time, administration route, excretion rate, treatment period, and simultaneously used drugs, and other factors well known in the medical field. For example, the pharmaceutical composition of the present invention may be administered at a dose of 1 mg/kg to 200 mg/kg, preferably 1 mg/kg to 50 mg/kg once a day or three times a day in divided doses. The dose is not intended to limit the scope of the present invention in any way.

The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects in consideration of all the aforementioned factors, and this amount may be easily determined by a person skilled in the art.

Still another aspect of the present invention provides a food composition for adjuvant anticancer treatment for suppressing the anticancer drug resistance of cancer cells occurring in a low-carbohydrate state, containing one or more materials selected from the group consisting of curcumin, quercetin, catechins, resveratrol, antocyanins, isoflavones, or derivatives thereof.

Since the physiologically active material having HIF-1α inhibitory activity as described in the composition for suppressing angiogenesis described above, and the like are used in the food composition for adjuvant anticancer treatment of the present invention, the description of the contents commonly shared between the two is omitted in order to avoid excess complexity of the present specification.

The functional food composition may be ingested with a low-carbohydrate diet to increase the therapeutic effect of anticancer agents, and may be used separately or simultaneously with therapeutic agents before or after the onset of the disease. The low-carbohydrate diet preferably means the reduction of the intake of carbohydrates, which account for about 60% of the energy production rate in an average person's diet, to 20% or less of the energy production from carbohydrates based on the total energy production amount of food. Meanwhile, the food may mean a single food, but refers to all the food provided when more than one food is provided in the form of a meal.

The functional food composition of the present invention may be used alone or used together with other foods or food ingredients, and may be appropriately used according to a typical method. The mixing amount of the physiologically active ingredient may be suitably determined depending on its purpose of use (for prevention or amelioration). Generally, the physiologically active ingredient may be added in an amount of 15% by weight or less, preferably 0.0001 to 10% by weight, based on the total weight of the composition of the present invention during the production of food or beverage, but in the case of long-term intake for health and hygiene purposes or for health regulation purposes, the amount may be equal to or less than the above range.

In particular, when the physiologically active material of the present invention is a natural product-based processed product, an appropriate amount that can exhibit an effect similar or equivalent to that of a natural product may be added, and the blood concentration of the ingested physiologically active material may be used as a measure for calculating such an appropriate amount.

In addition to containing the above active ingredient as an essential ingredient, the functional food composition of the present invention may contain carbohydrate food (cereals, potatoes, nuts, sugar, taffies, and the like), protein food (meats, eggs, milk, seafood, soybeans, peanuts, and the like), fatty food (vegetable oils, animal oils, margarines, and the like) and the like. Furthermore, the functional food composition of the present invention may additionally contain vitamin food (green and yellow vegetables, fruits, liver, seafood, and the like), inorganic food (small fish eaten with bones, seaweed, salt, milk, and the like) as needed.

The functional food composition of the present invention may contain various nutrients, vitamins, minerals (electrolytes), flavoring agents such as synthetic flavoring agents and natural flavoring agents, colorants and fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in a carbonated beverage, or the like, in addition to the above-described additives. These ingredients may be used either alone or in combinations thereof. The proportion of these nutrients and the like may also be appropriately selected by a person skilled in the art.

### Advantageous Effects

The composition of the present invention can suppress angiogenesis and overcome the resistance of cancer cells occurring in a low-carbohydrate state, and thus can be usefully used for angiogenesis suppression and adjuvant anticancer treatment.

### DESCRIPTION OF DRAWINGS

FIG. 1 illustrates the results of measuring intracellular pH by culturing breast cancer cells at normal or low glucose concentrations and treating breast cancer cells with an anticancer drug and an intracellular acidity regulator.

FIG. 2 illustrates the results of measuring intracellular pH by culturing breast cancer cells at normal or low glucose concentrations and treating breast cancer cells with an anticancer drug and curcumin.

FIG. 3 illustrates the cell viability after culturing breast cancer cells at normal or low glucose concentrations and co-treating breast cancer cells with an anticancer drug and an intracellular acidity regulator or curcumin.

FIG. 4 illustrates the results of confirming the degree of anticancer drug resistance after culturing colorectal cancer cells at normal or low glucose concentrations, co-treating colorectal cancer cells with an anticancer drug and an intracellular acidity regulator.

FIG. 5 illustrates the results of confirming that the effect of the co-treatment of an anticancer drug and an intracellular acidity regulator is caused by HIF-1 expression suppression.

### MODES OF THE INVENTION

Hereinafter, one or more specific exemplary embodiments will be described in more detail through examples. However, these examples are provided only for exemplarily explaining the one or more specific exemplary embodiments, and the scope of the present invention is not limited to these examples.

### Example 1: Effect of Changes in Intracellular Acidity Under Low-Carbohydrate Conditions

Breast cancer cells were cultured at a normal glucose (GLU) concentration of 10 nM or a low concentration of 5 nM, and intracellular acidity was confirmed with or without the treatment of paclitaxel (PTX), which is an anticancer drug, and cariporide (CAR), which is a drug that regulates intracellular acidity.

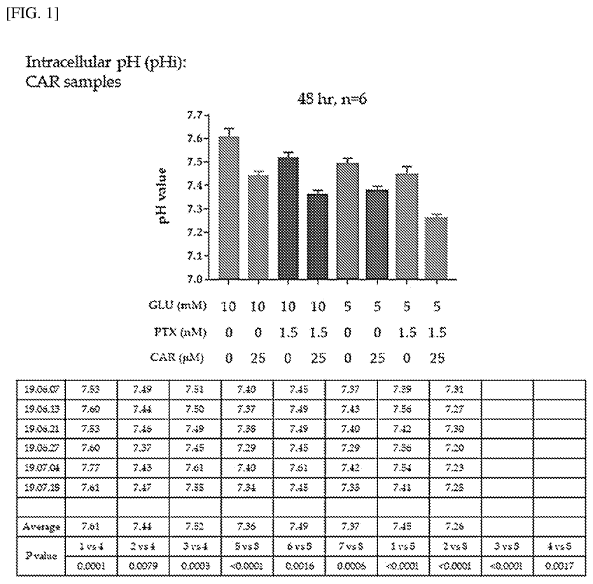

As a result, when the cancer cells were in a low-carbohydrate state, it was confirmed that intracellular acidity was closest to normal in an experimental group in which the anticancer drug and cariporide were treated in combination (FIG. 1).

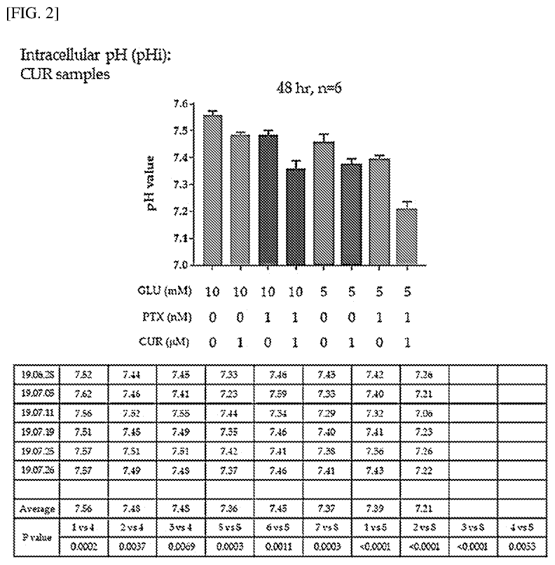

In addition, as a result of performing the same experiment by replacing cariporide with curcumin (CUR), it was confirmed that when the cancer cells were in a low-carbohydrate state, intracellular acidity was closest to normal in the experimental group in which the anticancer drug and curcumin were treated in combination (FIG. 2).

### Example 2: Changes in Viability of Cancer Cells by Regulation of Intracellular Acidity

Breast cancer cells were cultured at a normal glucose (GLU) concentration of 10 nM or a low concentration of 5 nM, and cell viability was confirmed with and without the treatment of paclitaxel (PTX), which is an anticancer drug, and cariporide (CAR) or curcumin (CUR).

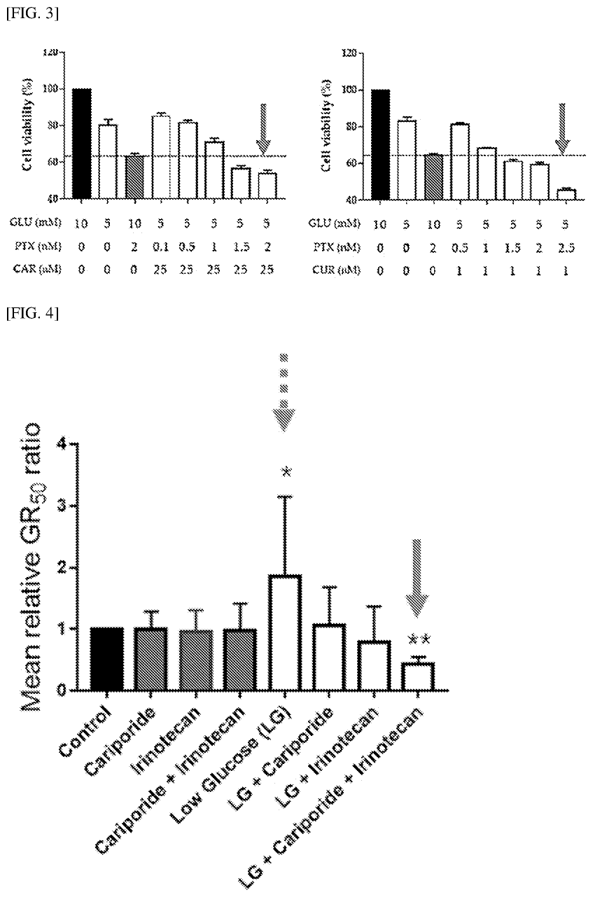

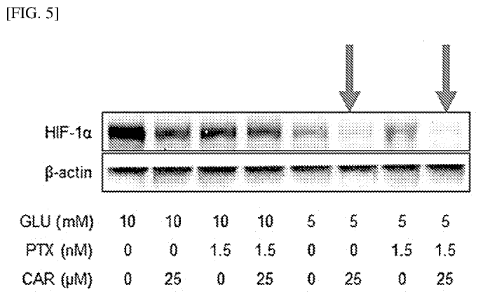

As a result, when the cancer cells were in a low-carbohydrate state, it was confirmed that the experimental group in which cariporide or curcumin was treated in combination with the anticancer drug had the best anticancer effect (FIG. 3).

Furthermore, colorectal cancer cells were cultured at a high glucose concentration (10 nM) or a low glucose concentration (5 nM), and it was confirmed by a growth rate 50 ratio (GR50 ratio) while treating irinotecan, which is an anticancer drug, and cariporide in combination whether the cancer cells had anticancer drug resistance.

As a result of confirmation, it was confirmed that when the cancer cells were cultured, anticancer drug resistance was increased as long as the low-carbohydrate state was maintained, whereas the treatment of the anticancer drug and cariporide in combination can overcome anticancer drug resistance and significantly reduce cell viability (FIG. 4).

### Example 3: Confirmation of Mechanism of Increase in Anticancer Effect

A mechanism by which the anticancer effect of anticancer drug is increased by cariporide or curcumin in a low-carbohydrate state was studied.

Breast cancer cells were cultured at a normal glucose (GLU) concentration of 10 nM or a low concentration of 5 nM, and changes in the expression of hypoxia-inducible factor-1 alpha (HIF-1α) were confirmed with and without the treatment of paclitaxel (PTX), which is an anticancer drug, and cariporide (CAR).

As a result, it was confirmed that the combined effect of the anticancer drug and the intracellular acidity-regulating material mainly induces intracellular hypoxia, thereby suppressing the expression of HIF-1α, which aids cell proliferation.

Through the results up to now, it can be seen that when curcumin, and a similar flavonoid, resveratrol, and the like are included in a processed food such as a beverage and a powder with a carbohydrate content of 20% or less of total energy, and the processed food is administered in combination with an anticancer drug, anticancer drug resistance can be overcome and the anticancer effect can be increased.

1.-9. (canceled)

10. A method for suppressing angiogenesis and NHE1 in cancer patients, the method comprising administering to the cancer patients a composition comprising one or more materials selected from the group consisting of curcumin, quercetin, catechins, resveratrol, antocyanins, isoflavones, or derivatives thereof.

11. The method of claim 10, wherein the composition inhibits hypoxia-inducible factor-1 alpha (HIF-1α).

12. The method of claim 10, wherein the composition is administered in combination with an anticancer drug.

13. The method of claim 12, wherein the anticancer drug is selected from the group consisting of irinotecan, paclitaxel, doxorubicin, cisplatin, docetaxel, cabazitaxel, carboplatin, etoposide, gemcitabine, 5-fluorouracil, capecitabine, gefitinib, sorafenib, and imatinib.

14. The method of claim 10, wherein the composition is administered in combination with a low-carbohydrate diet.

15. A food composition for adjuvant anticancer treatment for suppressing the anticancer drug resistance of cancer cells occurring in a low-carbohydrate state, containing one or more materials selected from the group consisting of curcumin, quercetin, catechins, resveratrol, antocyanins, isoflavones, or derivatives thereof.

16. A method for suppressing the resistance of cancer cells to a low-carbohydrate diet in cancer patients, the method comprising administering to the cancer patients a composition comprising one or more materials selected from the group consisting of curcumin, quercetin, catechins, resveratrol, antocyanins, isoflavones, or derivatives thereof.

17. The method of claim 10, wherein the composition inhibits hypoxia-inducible factor-1 alpha (HIF-1α).

18. The method of claim 10, wherein the composition is administered in combination with an anticancer drug.

19. The method of claim 12, wherein the anticancer drug is selected from the group consisting of irinotecan, paclitaxel, doxorubicin, cisplatin, docetaxel, cabazitaxel, carboplatin, etoposide, gemcitabine, 5-fluorouracil, capecitabine, gefitinib, sorafenib, and imatinib.

20. The method of claim 10, wherein the composition is administered in combination with a low-carbohydrate diet.
